(19) United States
(12) Patent Application Publication
(10) Pub. No.: US 2019/0246686 A1
(43) Pub. Date: Aug. 15, 2019

(54) Alternative Nicotine Carriers for Solid Products

(71) Applicant: Altria Client Services LLC, Richmond, VA (US)

(72) Inventors: Shuzhong Zhuang, Glen Allen, VA (US); Diane L. Gee, Chesterfield, VA (US)

(73) Assignee: Altria Client Services LLC, Richmond, VA (US)

(21) Appl. No.: 16/274,861

(22) Filed: Feb. 13, 2019

Related U.S. Application Data
(60) Provisional application No. 62/631,289, filed on Feb. 15, 2018.

(51) Int. Cl. A24B 15/16; A23G 4/06; A23G 4/08; A23G 4/04

(52) U.S. Cl. CPC A24B 15/16; A23G 4/068; A23G 4/064; C07D 401/04; A23G 4/043; A23G 2200/14; A23G 4/08

(57) ABSTRACT

This document provides methods and systems for stabilizing nicotine and incorporating nicotine into one or more oral products. This document also provides oral products. Nicotine can be stabilized by mixing nicotine with a silicon oxide-containing porous solid such that the nicotine absorbs into pores of the porous solid to form a porous solid-nicotine mixture. In some cases, a porous solid-nicotine mixture can be combined with one or more binders and molded into an oral product.

### CROSS REFERENCE TO RELATED APPLICATIONS

This application claims the benefit of U.S. Provisional Application No. 62/631,289, filed Feb. 15, 2018, which is incorporated by reference herein in its entirety.

### TECHNICAL FIELD

Methods and systems for incorporating nicotine into oral products are described. For example, methods and systems provided herein can stabilize nicotine for handling and/or incorporation into an oral product.

### BACKGROUND

Nicotine is a compound found naturally in tobacco plants. Various methods and systems have been developed for providing nicotine to adult tobacco consumers without the presence of tobacco plant tissue. Some ways tobacco-free nicotine is provided include transdermal patches, lozenges, and nicotine chewing gums.

Nicotine, or 3-(1-methyl-2-pyrrolidinyl)pyridine, is a tertiary amine with the following structure:

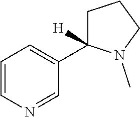

Under ambient conditions, nicotine is an oily, volatile, hygroscopic compound that is sensitive to light and air. Chemical and physical properties of nicotine present a number of processing and stability issues. For example, because nicotine is volatile, it may evaporate during its incorporation into an oral product such as a gum or lozenge. As another example, some nicotine products may require added safety gear and protocols for processing and handling. In an effort to reduce potential processing and stability issues associated with the nicotine compound, a number of nicotine complexes have been developed. For example, one method includes the preparation of a complex of nicotine and an ion exchange resin. A well-known complex that is currently used in the commercially-available nicotine chewing gums is nicotine polacrilex, which is a complex of nicotine and the cation exchange resin AMBERLITE 164. Another method for creating nicotine complexes includes absorbing nicotine into pores of cellulosic fibers.

### SUMMARY

This document provides methods and systems for stabilizing nicotine and incorporating nicotine into an oral product. This document provides oral products incorporating nicotine. In some cases, an oral product provided herein can include a silicon oxide-containing porous solid and nicotine absorbed into pores of the silicon oxide-containing porous solid. In some cases, an oral product provided herein can include a binder matrix, a silicon oxide-containing porous solid within the binder matrix, and nicotine absorbed into pores of the silicon oxide-containing porous solid. Methods and systems provided herein include mixing nicotine with a silicon oxide-containing porous solid to produce a porous solid-nicotine mixture. In some cases, the porous solid-nicotine mixture can be combined with one or more binders and the mixture can be molded into an oral product having a binder matrix.

Direct incorporation of nicotine into oral products can present a number of difficulties. In some cases, mixing nicotine with a mixture of dry ingredients can disrupt certain molding processes, such as compression molding. In some cases, the direct incorporation of nicotine can result in an excessively fast release rate from the resulting oral product. Nicotine complexes, such as nicotine polacrilex, however, can present problems with incorporating nicotine into an oral product. For example, certain molding processes can use temperatures that cause certain nicotine complexes to degrade. In some cases, nicotine complexes can result in an excessively slow release rate of nicotine from the resulting oral product. Moreover, the release rate can be rate limited by chemical reactions that allow the nicotine to be released, thus an adult tobacco consumer can have a limited ability to adjust the release of nicotine. Nicotine complexes sometimes produce acid by-products during the release of nicotine, which can further impede the release of nicotine and/or produce an unpleasant flavor. Some oral products incorporating nicotine complexes can incorporate buffers to control the release rate and/or counteract the release of acid by the nicotine complex, but these buffers can provide unpleasant flavors. For example, sodium carbonate and/or sodium bicarbonate can be used as a buffering agent with a nicotine complex, but sodium carbonate and/or sodium bicarbonate can also provide an undesirable or off-taste. Other nicotine complexes have low absorption rates of nicotine.

The products and methods described herein can provide several advantages. First, combining nicotine with a silicon oxide-containing porous solid as provided herein can provide stabilized nicotine that can be used as an oral product alone or incorporated into oral products. In some cases, oral products provided herein include a binder matrix, a silicon oxide-containing porous solid dispersed in the binder matrix, and nicotine absorbed in pores of a silicon oxide-containing porous solid. The porous solid-nicotine combination provided herein can be used in a wide variety of molding operations, including compression molding techniques that call for dry ingredients.

Second, combining nicotine with a silicon oxide-containing porous solid as provided herein can improve the ease of handling and processability of nicotine. For example, nicotine absorbed in a silicon oxide-containing porous solid as described herein may exhibit limited release rates or require a solvent for release of the nicotine such that processing restrictions and protective equipment may not be necessary.

Third, combining nicotine with a silicon oxide-containing porous solid as provided herein can provide greater absorption of nicotine as compared to other nicotine complexes. For example, some nicotine complexes, such as nicotine-cellulose complexes, can have maximum nicotine absorption capacities of less than 10 mL per 100 g. However, the compositions and methods described herein can provide a water capacity or nicotine capacity of 20 mL per 100 grams, 35 mL per 100 grams, 40 mL per 100 grams, 45 mL per 100 grams, or greater.

Fourth, combining nicotine with a silicon oxide-containing porous solid as provided herein can provide a wide range of release rates for nicotine in the oral products described herein.

Silicon oxide-containing porous solid-nicotine mixtures used in the methods, systems, and oral products provided herein can be naturally-derived or synthetic.

In one aspect, a composition is provided, the composition including nicotine and a silicon oxide-containing porous solid. In some embodiments, the porous solid is amorphous. In some embodiments, the nicotine is absorbed within pores of the porous solid. In some embodiments, the porous solid can comprise a material selected from a silica gel, a silica-containing polymer, an aluminosilicate, and combinations thereof. The aluminosilicate can be a natural or synthetic zeolite. In some embodiments, the nicotine can be selected from free base nicotine and a nicotine salt.

In some embodiments, the composition can optionally include one or more of the following features. The porous solid can comprise a plurality of particles. The plurality of particles has an average particle size of 10 μm or greater. The porous solid can have an average pore diameter of from about 2 nm to about 50 nm, from about 50 nm to about 300 nm, or from about 0.5 nm to about 2 nm. The porous solid can have a Brunauer-Emmett-Teller (BET) surface area of from about 1 m²/g to 1200 m²/g. The porous solid can have an average pore diameter of from 2 nm to 50 nm, from 50 nm to 300 nm, or from 0.5 nm to 2 nm. The porous solid can have a BET surface area of from 1 m²/g to 1200 m²/g. The porous solid can have a nicotine absorption capacity or a water absorption capacity of 2 mL per 100 grams to 50 mL per 100 grams.

In some embodiments, the composition can further include a diluent. The diluent can be selected from the group consisting of solvents, plasticizers, humectants, flavorants, and combinations thereof.

In another aspect, a method for stabilizing nicotine is provided, the method including mixing nicotine with a silicon oxide-containing porous solid such that the nicotine absorbs into pores of the porous solid to form a porous solid-nicotine mixture. In some embodiments, the porous solid can comprise a material selected from a silica gel, a silica-containing polymer, an aluminosilicate, and combinations thereof. The aluminosilicate can be a natural or synthetic zeolite.

In some embodiments, the porous solid used in the method can optionally include one or more of the following features. The porous solid can comprise a plurality of particles. The plurality of particles has an average particle size of 10 μm or greater. The porous solid can have an average pore diameter of from about 2 nm to about 50 nm, from about 50 nm to about 300 nm, or from about 0.5 nm to about 2 nm. The porous solid can have a Brunauer-Emmett-Teller (BET) surface area of from about 1 m²/g to 1200 m²/g. The porous solid can have an average pore diameter of from 2 nm to 50 nm, from 50 nm to 300 nm, or from 0.5 nm to 2 nm. The porous solid can have a BET surface area of from 1 m²/g to 1200 m²/g. The porous solid can have a nicotine absorption capacity or a water absorption capacity of 2 mL per 100 grams to 50 mL per 100 grams.

In some embodiments of the method, the nicotine can be selected from free base nicotine and a nicotine salt. The nicotine can comprise at least 1 weight percent nicotine. The method can optionally further include diluting nicotine with a diluent prior to mixing the nicotine with the porous solid. In some embodiments, the diluent is selected from the group consisting of solvents, plasticizers, humectants, flavorants, and combinations thereof. In some embodiments, the nicotine can comprise between 2 weight percent and 75 weight percent nicotine and at least one diluent. In some embodiments, the nicotine can comprise between 2 weight percent and 95 weight percent nicotine and at least one diluent.

The method can optionally further include allowing the porous solid-nicotine mixture to equilibrate within a sealed container for at least 1 hour.

In another aspect, a method for incorporating nicotine into an oral product is provided, the method including: (a) mixing nicotine with a silicon oxide-containing porous solid to produce a porous solid-nicotine mixture; (b) mixing the porous solid-nicotine mixture with one or more binders to form an oral product pre-molding mixture; and (c) molding the oral product pre-molding mixture into an oral product. In some embodiments of the method, the ratio of nicotine to porous solid by weight can be between 1:1000 and 10:1. In some embodiments of the method, the ratio of nicotine to porous solid by weight can be between 1:1000 and 1:1. In some embodiments of the method, the ratio of nicotine to porous solid by weight can be between 1:100 and 1:1. In some embodiments of the method, the ratio of nicotine to porous solid by weight can be between 1:10 and 1:1. In some embodiments of the method, the ratio of nicotine to porous solid by weight is greater than 1:1000. In some embodiments of the method, the ratio of nicotine to porous solid by weight is greater than 1:100. In some embodiments of the method, the ratio of nicotine to porous solid by weight is less than 1:100. In some embodiments of the method, the ratio of nicotine to porous solid by weight is less than 1:1.

The method can optionally further include diluting the nicotine with one or more diluents prior to mixing the nicotine with the porous solid to form the porous solid-nicotine mixture. In some embodiments, the one or more diluents can include a solvent, a plasticizer, a flavorant, a humectant, or combinations thereof. In some embodiments, the one or more diluents comprise propylene glycol and the nicotine is diluted to a concentration of between 5% and 25% nicotine prior to mixing the nicotine with the porous solid to form the porous solid-nicotine mixture. In some embodiments, the one or more diluents comprise water and the nicotine is diluted to a concentration of between 5% and 95% nicotine prior to mixing the nicotine with the porous solid to form the porous solid-nicotine mixture. In some embodiments, the one or more diluents comprise ethyl alcohol and the nicotine is diluted to a concentration of between 5% and 25% nicotine prior to mixing the nicotine with the porous solid to form the porous solid-nicotine mixture.

In some embodiments, the method can further include holding the porous solid-nicotine mixture in a sealed container for at least an hour prior to mixing the porous solid-nicotine mixture with the binder such that the porous solid-nicotine mixture equilibrates. In some embodiments, the binder can be a chewing gum base. In some embodiments, the binder is selected from the group consisting of dextrin or dextrin derivative, carboxymethyl cellulose, hydroxypropyl cellulose, hydroxyethyl cellulose, hydroxypropyl methyl cellulose, methyl cellulose, starch, konjac, collagen, inulin, soy protein, whey protein, casein, wheat gluten, carrageenan, alginates, propylene glycol alginate, xanthan, dextrin, pullulan, curdlan, gellan, locust bean gum, guar gum, tara gum, gum tragacanth, pectin, agar, zein, karaya, gelatin, psyllium seed, chitin, chitosan, gum acacia, polyvinyl pyrrolidone, polyethylene oxide, polyvinyl alcohol, guar gum, xanthan, cellulose, maltodextrin or other modified starch, polyurethane, silicon polymer, polyester, polyacrylate, polyethylene, poly(styrene-ethylene-butylene-styrene) (“SEBS”), poly(styrene-butadiene-styrene) (“SBS”), poly(styrene-isoprene-styrene)(“SIS”), couma macrocarpa, loquat, tunu, jelutong, chicle, styrene-butadiene rubber, butyl rubber, and polyisobutylene, glycerol esters of gum, terpene resins, polyvinyl acetate, paraffin, microcrystalline wax, hydrogenated vegetable oils, lecithin, glycerol monosterate, natural latexes, chicle, spruce gum, mastic gum, or a combination thereof.

In some embodiments of the method, molding the oral product pre-molding mixture into the oral product can comprise compression molding the oral product pre-molding mixture into a predetermined shape. The oral product pre-molding mixture can optionally comprise a dry mixture of ingredients. The oral product pre-molding mixture can optionally be substantially free of ion-exchange resins. The oral product pre-molding mixture can optionally be substantially free of buffering agents.

In another aspect, an oral product is provided, including a mixture of nicotine and a silicon oxide-containing porous solid, the nicotine being absorbed into pores of the porous solid. In some embodiments, the oral product can further comprise a binder holding the mixture of porous solid and nicotine together into a solid piece. In some embodiments, the oral product can further comprise one or more plasticizers, one or more humectants, one or more flavorants, one or more sweeteners, one or more colorants, or a combination thereof. In some embodiments, the oral product can further comprise one or more gum bases. In some embodiments, the oral product can further comprise one or more soluble fibers. In some embodiments, the oral product can further comprise one or more insoluble fibers. In some embodiments, the oral product can further comprise a coating.

In some embodiments, the oral product can be selected from a compressed tablet, a chewable tablet, a dissolvable tablet, and combinations thereof. In some embodiments, the oral product can be chewing gum.

The details of one or more embodiments of the invention are set forth in the accompanying drawings and the description below. Other features, objects, and advantages of the invention will be apparent from the description and drawings, and from the claims.

### DETAILED DESCRIPTION

This document provides methods and systems related to stabilizing nicotine, incorporating nicotine into an oral product, and providing an oral product having desirable nicotine-release characteristics. Nicotine can be stabilized by mixing nicotine with a silicon oxide-containing porous solid such that the nicotine absorbs into pores of the porous solid to form a porous solid-nicotine mixture. An oral product can be manufactured by mixing a porous solid-nicotine mixture provided herein with one or more binders to form an oral product pre-molding mixture and molding the oral product pre-molding mixture into an oral product. Combining nicotine with a silicon oxide-containing porous solid as provided herein can provide stabilized nicotine that can be used in a wide variety of molding operations, including compression molding techniques that call for dry ingredients. An oral product provided herein can have desirable nicotine-release characteristics.

Nicotine used in the porous solid-nicotine mixture provided herein can be tobacco-derived nicotine, synthetic nicotine, or a combination thereof. Nicotine can be purchased from commercial sources, whether tobacco-derived or synthetic. Tobacco-derived nicotine can include one or more additional tobacco organoleptic components other than nicotine. The tobacco-derived nicotine can be extracted from raw (e.g., green leaf) tobacco and/or processed tobacco. Processed tobaccos can include fermented and unfermented tobaccos, dark air-cured, dark fire-cured, burley, flue cured, and cigar filler or wrapper, as well as the products from the whole leaf stemming operation. The tobacco can also be conditioned by heating, sweating and/or pasteurizing steps as described in U.S. Publication Nos. 2004/0118422 or 2005/0178398. Fermenting typically is characterized by high initial moisture content, heat generation, and a 10 to 20% loss of dry weight. See, e.g., U.S. Pat. Nos. 4,528,993; 4,660,577; 4,848,373; and 5,372,149. By processing the tobacco prior to extracting nicotine and other organoleptic components, the tobacco-derived nicotine may include other ingredients. The tobacco-derived nicotine can be obtained by mixing cured and fermented tobacco with water or another solvent (e.g., ethanol) followed by removing the insoluble tobacco material. The tobacco extract may be further concentrated or purified. In some cases, select tobacco constituents can be removed. Nicotine can also be extracted from tobacco in the methods described in the following patents: U.S. Pat. Nos. 2,162,738; 3,139,436; 3,396,735; 4,153,063; 4,448,208; and 5,487,792.

As used herein, “about” refers to +/−10%. For example, “about 100” would include 90 to 110.

Nicotine can be pure, substantially pure, or diluted prior to combination with a silicon oxide-containing porous solid. In some cases, nicotine is diluted to a concentration of between 1 weight percent and 75 weight percent prior to mixing the nicotine with the silicon oxide-containing porous solid. In some cases, nicotine is diluted to a concentration of between 1 weight percent and 95 weight percent prior to mixing the nicotine with the silicon oxide-containing porous solid. In some cases, nicotine is diluted to a concentration of between 2 weight percent and 95 weight percent prior to mixing the nicotine with the silicon oxide-containing porous solid. In some cases, nicotine is diluted to a concentration of between 2 weight percent and 50 weight percent prior to mixing the nicotine with the silicon oxide-containing porous solid. In some cases, nicotine is diluted to a concentration of between 5 weight percent and 25 weight percent prior to mixing the nicotine with the silicon oxide-containing porous solid. In some cases, nicotine is diluted to a concentration of less than 95 weight percent prior to mixing the nicotine with the silicon oxide-containing porous solid. In some cases, nicotine is diluted to a concentration of less than 75 weight percent prior to mixing the nicotine with the silicon oxide-containing porous solid. In some cases, nicotine is diluted to a concentration of less than 50 weight percent prior to mixing the nicotine with the silicon oxide-containing porous solid. In some cases, nicotine is diluted to a concentration of less than 25 weight percent prior to mixing the nicotine with the silicon oxide-containing porous solid. For example, nicotine can be diluted to a concentration of about 10 weight percent prior to mixing the nicotine with the silicon oxide-containing porous solid.

In some cases, an oral product including a porous solid-nicotine mixture provided herein can include between 0.1 mg of nicotine per portion and 10.0 mg of nicotine per portion. In some cases, an oral product including a porous solid-nicotine mixture provided herein includes between 1.0 mg of nicotine per portion and 6.0 mg of nicotine per portion. In some cases, an oral product including a porous solid-nicotine mixture provided herein comprises at least 0.1 mg of nicotine per portion. In some cases, an oral product including a porous solid-nicotine mixture provided herein comprises at least 0.5 mg of nicotine per portion. In some cases, an oral product including a porous solid-nicotine mixture provided herein comprises at least 1.0 mg of nicotine per portion. In some cases, an oral product including a porous solid-nicotine mixture provided herein comprises at least 5.0 mg of nicotine per portion. In some cases, an oral product including a porous solid-nicotine mixture provided herein comprises less than 10.0 mg of nicotine per portion. In some cases, an oral product including a porous solid-nicotine mixture provided herein comprises less than 7.5 mg of nicotine per portion. In some cases, an oral product including a porous solid-nicotine mixture provided herein comprises less than 6.0 mg of nicotine per portion. In some cases, an oral product including a porous solid-nicotine mixture provided herein comprises less than 5.0 mg of nicotine per portion. In some cases, an oral product including a porous solid-nicotine mixture provided herein comprises less than 1.0 mg of nicotine per portion.

In some embodiments, a method for stabilizing nicotine is provided, comprising mixing nicotine with a silicon oxide-containing porous solid such that the nicotine absorbs into pores of the porous solid to form a porous solid-nicotine mixture. In some embodiments, a method of stabilizing nicotine can include absorbing from about 0.1 mg nicotine to about 50.0 mg nicotine per 100.0 mg silicon oxide-containing porous solid. In some embodiments, a method of stabilizing nicotine can include absorbing from 0.1 mg nicotine to 50.0 mg nicotine per 100.0 mg silicon oxide-containing porous solid. In some embodiments, a method of stabilizing nicotine can include absorbing at least 0.1 mg nicotine per 100.0 mg silicon oxide-containing porous solid. In some embodiments, a method of stabilizing nicotine can include absorbing at least 1.0 mg nicotine per 100.0 mg silicon oxide-containing porous solid. In some embodiments, a method of stabilizing nicotine can include absorbing at least 5.0 mg nicotine per 100.0 mg silicon oxide-containing porous solid. In some embodiments, a method of stabilizing nicotine can include absorbing at least 10.0 mg nicotine per 100.0 mg silicon oxide-containing porous solid. In some embodiments, a method of stabilizing nicotine can include absorbing at least 20.0 mg nicotine per 100.0 mg silicon oxide-containing porous solid. In some embodiments, a method of stabilizing nicotine can include absorbing at least 30.0 mg nicotine per 100.0 mg silicon oxide-containing porous solid. In some embodiments, a method of stabilizing nicotine can include absorbing at least 40.0 mg nicotine per 100.0 mg silicon oxide-containing porous solid. In some embodiments, a method of stabilizing nicotine can include absorbing at least 50.0 mg nicotine per 100.0 mg silicon oxide-containing porous solid. In some embodiments, the porous solid-nicotine mixture can be used for processing nicotine, e.g., transferring nicotine to one or more nicotine adsorbents, transferring nicotine to one or more products, or incorporation of nicotine into one or more products, and the like.

The nicotine can optionally be diluted prior to mixing the nicotine with the silicon oxide-containing porous solid. Nicotine can be diluted with any suitable diluent. Diluting the nicotine can provide more liquid volume for the nicotine to help meter a precise amount of nicotine. Diluents can also facilitate absorption of nicotine into a silicon oxide-containing porous solid. In some cases, the diluent can be one or more plasticizers, one or more humectants, one or more flavorants, or a combination thereof. In some cases, a single substance can serve as both a plasticizer and a humectant, both a humectant and a flavorant, both a plasticizer and a flavorant, or as all three. For example, propylene glycol can serve as both a plasticizer and a humectant. For example, honey can serve as both a humectant and a flavorant. In some cases, the diluent can include a solvent (e.g., ethanol, water, or any other polar solvent generally recognized as safe (“GRAS”) for human consumption). In some cases, ethanol can be used as a diluent. Ethanol can act as a solvent, but also provide some plasticizing characteristics in the methods, systems, and products provided herein. In some cases, the diluent can include a sweetener. In some cases, the diluent can include a combination of plasticizers, humectants, solvents, sweeteners, and/or flavorants such that the porous solid-nicotine mixture mimics the flavor profile and tactile experience of certain tobacco products.

Suitable plasticizers include propylene glycol, glycerin, vegetable oil, partially hydrogenated vegetable oil, and medium chain triglycerides. In some cases, the plasticizer can include phthalates. In some embodiments, the plasticizer can act as a solvent for nicotine, while in other embodiments, the plasticizer can be miscible with a nicotine solvent. Esters of polycarboxylic acids with linear or branched aliphatic alcohols of moderate chain length can also be used as plasticizers. In addition to serving as a diluent, plasticizers can facilitate the molding processes described below. Plasticizers can, in some cases, soften an oral product. In some cases, an oral product can include up to 20 weight percent plasticizer. In some cases, an oral product can include up to 15 weight percent plasticizer. In some cases, an oral product can include up to 10 weight percent plasticizer. In some cases, an oral product can include up to 9 weight percent plasticizer. In some cases, an oral product can include up to 8 weight percent plasticizer. In some cases, an oral product can include up to 7 weight percent plasticizer. In some cases, an oral product can include up to 6 weight percent plasticizer. In some cases, an oral product can include up to 5 weight percent plasticizer. In some cases, an oral product can include up to 4 weight percent plasticizer. In some cases, an oral product can include up to 3 weight percent plasticizer. In some cases, an oral product can include up to 2 weight percent plasticizer. In some cases, an oral product can include up to 1 weight percent plasticizer. In some cases, an oral product can include up to 0.5 weight percent plasticizer. In some cases, an oral product includes between 0.5 and 10 weight percent plasticizer, between 1 and 8 weight percent plasticizer, or between 2 and 4 weight percent plasticizer. For example, an oral product can include about 3 to 6.5 weight percent of propylene glycol.

A humectant is a substance that is used to keep things moist. Humectants can be hygroscopic. Suitable humectants include propylene glycol, hexylene glycol, butylene glycol, glyceryl triacetate, vinyl alcohol, neoagarobiose, sugar polyols (such as glycerol, sorbitol (E420), xylitol, maltitol, mannitol, and isomalt), polymeric polyols (e.g., polydextrose), quillaia, alpha hydroxyl acids (e.g., lactic acid), glycerin, aloe vera gel, and honey.

Flavorants can be natural or artificial. Flavorants can be selected from, e.g., licorice, wintergreen, cherry and berry type flavorants, Drambuie, bourbon, scotch, whiskey, spearmint, peppermint, lavender, cinnamon, cardamon, apium graveolents, clove, cascarilla, nutmeg, sandalwood, bergamot, geranium, honey essence, rose oil, vanilla, lemon oil, orange oil, Japanese mint, cassia, caraway, cognac, jasmin, chamomile, menthol, ylangylang, sage, fennel, pimento, ginger, anise, coriander, coffee, mint oils from a species of the genus Mentha, cocoa, and combinations thereof. Synthetic flavorants can also be used. In certain embodiments, a combination of flavorants can be combined to imitate a tobacco flavor. The particular combination of flavorants can be selected from flavorants that are GRAS.

A variety of synthetic and/or natural sweeteners can be used as the diluent or added separately to an oral product. Suitable natural sweeteners include sugars, for example, monosaccharides, disaccharides, and/or polysaccharide sugars, and/or mixtures of two or more sugars. In some cases, a diluent can include one or more of the following: sucrose or table sugar; honey or a mixture of low molecular weight sugars not including sucrose; glucose or grape sugar or corn sugar or dextrose; molasses; corn sweetener; corn syrup or glucose syrup; fructose or fruit sugar; lactose or milk sugar; maltose or malt sugar or maltobiose; sorghum syrup; mannitol or manna sugar; sorbitol or d-sorbite or d-sorbitol; fruit juice concentrate; and/or mixtures or blends of one or more of these ingredients. A diluent can, in some cases, include non-nutritive sweeteners. Suitable non-nutritive sweeteners include stevia, saccharin; aspartame; sucralose; or acesulfame potassium.

Silicon oxide-containing porous solids used in the methods, systems, and oral products provided herein can be naturally-derived or synthetic. In some cases, silicon oxide-containing porous solids used in the methods, systems, and oral products provided herein can include silica gel. Silicon oxide-containing porous solids used in the methods, systems, and oral products provided herein can further include silica polymers. In some cases, silicon oxide-containing porous solids used in the methods, systems, and oral products provided herein can include aluminosilicate materials, such as natural or synthetic zeolites. Silicon oxide-containing porous solids used in the methods, systems, and oral products provided herein can include a plurality of particles having a variety of dimensions. In some cases, silicon oxide-containing porous solids used in the methods, systems, and oral products provided herein can include one or more silicon oxide-containing porous solids that are GRAS for human consumption. Silicon oxide-containing porous solids used in the methods, systems, and oral products provided herein can include food grade silicon oxide-containing porous solids, such as food grade amorphous silica gel.

The dimensions of the silicon oxide-containing porous solids used in the methods, systems, and oral products provided herein can (in addition to the amount) impact the nicotine carrying capabilities of the silicon oxide-containing porous solids, processing of the nicotine or nicotine-porous solid complexes, and uniformity of nicotine distribution in the silicon oxide-containing porous solids, as well as the release characteristics of nicotine from the mixture and from an oral product provided herein. These characteristics, such as release profile of nicotine from an oral product, can be impacted by one or more of the porosity, Brunauer-Emmett-Teller (BET) surface area, and the amounts of nicotine absorbed in the silicon oxide-containing porous solids. The amount of nicotine absorbed in the silicon oxide-containing porous solid can also affect the nicotine release profile. In some embodiments, the nicotine can be absorbed into the silicon oxide-containing porous solid at from about 1% to about 100% of the nicotine absorption capacity of the silicon oxide-containing porous solid. In some embodiments, the nicotine can be absorbed into the silicon oxide-containing porous solid at from 1% to 100% of the nicotine absorption capacity of the silicon oxide-containing porous solid. In some embodiments, the nicotine can be absorbed into the silicon oxide-containing porous solid from 100% of the nicotine absorption capacity of the silicon oxide-containing porous solid, from at least 80% of the nicotine absorption capacity of the silicon oxide-containing porous solid, from at least 50% of the nicotine absorption capacity of the silicon oxide-containing porous solid, or from at least 10% of the nicotine absorption capacity of the silicon oxide-containing porous solid.

Silicon oxide-containing porous solids used in the methods, systems, and oral products provided herein can have pores with average pore sizes that range from between 0.5 nanometers to 300 nanometers. In an aspect, pore size is measured as the diameter of the pore. In some cases, silicon oxide-containing porous solids provided herein have pore sizes that range from between 10 nanometers to 200 nanometers, 2 nanometers to 50 nanometers, 50 nanometers to 300 nanometers, or 0.5 nanometers to 2 nanometers. In some cases, silicon oxide-containing porous solids provided herein have average pore sizes of at least 0.5 nanometers. In some cases, silicon oxide-containing porous solids provided herein have average pore sizes of at least 1 nanometers. In some cases, silicon oxide-containing porous solids provided herein have average pore sizes of at least 2 nanometers. In some cases, silicon oxide-containing porous solids provided herein have average pore sizes of at least 5 nanometers. In some cases, silicon oxide-containing porous solids provided herein have average pore sizes of at least 10 nanometers. In some cases, silicon oxide-containing porous solids provided herein have average pore sizes of at least 20 nanometers. In some cases, silicon oxide-containing porous solids provided herein have average pore sizes of at least 30 nanometers. In some cases, silicon oxide-containing porous solids provided herein have average pore sizes of at least 40 nanometers. In some cases, silicon oxide-containing porous solids provided herein have average pore sizes of at least 50 nanometers. In some cases, silicon oxide-containing porous solids provided herein have average pore sizes of at least 60 nanometers. In some cases, silicon oxide-containing porous solids provided herein have average pore sizes of at least 70 nanometers. In some cases, silicon oxide-containing porous solids provided herein have average pore sizes of at least 80 nanometers. In some cases, silicon oxide-containing porous solids provided herein have average pore sizes of at least 90 nanometers. In some cases, silicon oxide-containing porous solids provided herein have average pore sizes of at least 100 nanometers. In some cases, silicon oxide-containing porous solids provided herein have average pore sizes that range from between 20 nanometers to 100 nanometers. When mixing nicotine with silicon oxide-containing porous solids, nicotine can, in some embodiments, become absorbed into the pores in the silicon oxide-containing porous solids and held there by, e.g., in some embodiments, van der Waals forces. The number, sizes and size distribution, and chemical and physical surface properties of the pores can impact the release rate of nicotine incorporated into silicon oxide-containing porous solids and into an oral product. In some instances, the porous solids can have a Brunauer-Emmett-Teller (BET) surface area ranging from about 1 m²/g to about 1200 m²/g. In some instances, the porous solids can have a BET surface area ranging from 1 m²/g to 1200 m²/g. In some instances, the porous solids have a BET surface area of at least 1 m²/g. In some instances, the porous solids have a BET surface area of at least 10 m²/g. In some instances, the porous solids have a BET surface area of at least 50 m²/g. In some instances, the porous solids have a BET surface area of at least 100 m²/g. In some instances, the porous solids have a BET surface area of at least 250 m²/g. In some instances, the porous solids have a BET surface area of at least 500 m²/g. In some instances, the porous solids have a BET surface area of at least 750 m²/g. In some instances, the porous solids have a BET surface area of at least 1000 m²/g. In some instances, the porous solids have a BET surface area of less than 1200 m²/g. In some embodiments, the silicon oxide-containing porous solids can be microporous. In some embodiments, the silicon oxide-containing porous solids can be mesoporous. In some embodiments, the silicon oxide-containing porous solids can be macroporous.

The silicon oxide-containing porous solids can provide a nicotine capacity of 5 mL per 100 grams, 10 mL per 100 grams, 20 mL per 100 grams, 35 mL per 100 grams, 40 mL per 100 grams, 45 mL per 100 grams, or greater, depending on the porosity of the silicon oxide-containing porous solids. In some embodiments, silicon oxide-containing porous solids comprise a nicotine capacity of at least 1 mL per 100 grams. In some embodiments, silicon oxide-containing porous solids comprise a nicotine capacity of at least 5 mL per 100 grams. In some embodiments, silicon oxide-containing porous solids comprise a nicotine capacity of at least 10 mL per 100 grams. In some embodiments, silicon oxide-containing porous solids comprise a nicotine capacity of at least 20 mL per 100 grams. In some embodiments, silicon oxide-containing porous solids comprise a nicotine capacity of at least 35 mL per 100 grams. In some embodiments, silicon oxide-containing porous solids comprise a nicotine capacity of at least 40 mL per 100 grams. In some embodiments, silicon oxide-containing porous solids comprise a nicotine capacity of at least 45 mL per 100 grams. In some embodiments, silicon oxide-containing porous solids comprise a nicotine capacity of at least 50 mL per 100 grams. In some embodiments, the silicon oxide-containing porous solids can provide a nicotine capacity of from about 2 mL per 100 grams to about 50 mL per 100 grams, from about 10 mL per 100 grams to about 45 mL per 100 grams, from about 10 mL per 100 grams to about 40 mL per 100 grams, from about 10 mL per 100 grams to about 35 mL per 100 grams, from about 20 mL per 100 grams to about 35 mL per 100 grams, about 10 mL per 100 grams, about 15 mL/100 g, about 20 mL per 100 grams, about 25 mL per 100 grams, about 30 mL per 100 grams, or about 35 mL per 100 grams. In some embodiments, the silicon oxide-containing porous solids can provide a nicotine capacity of from 2 mL per 100 grams to 50 mL per 100 grams, from 10 mL per 100 grams to 45 mL per 100 grams, from 10 mL per 100 grams to 40 mL per 100 grams, from 10 mL per 100 grams to 35 mL per 100 grams, from 20 mL per 100 grams to 35 mL per 100 grams, 10 mL per 100 grams, 15 mL/100 g, 20 mL per 100 grams, 25 mL per 100 grams, 30 mL per 100 grams, or 35 mL per 100 grams. In some embodiments, the capacity of the silicon oxide-containing porous solids can be calculated using the BET surface area of the silicon oxide-containing porous solids. In some embodiments, the capacity of the silicon oxide-containing porous solids can be determined based on the water capacity of the silicon oxide-containing porous solids, in order to avoid difficulties that may be encountered in direct measurement of nicotine capacity. The silicon oxide-containing porous solids can provide a water capacity of 10 mL per 100 grams, 20 mL per 100 grams, 35 mL per 100 grams, 40 mL per 100 grams, 45 mL per 100 grams, 50 mL per 100 grams, or greater, depending on the porosity of the silicon oxide-containing porous solids. In some embodiments, silicon oxide-containing porous solids comprise a water capacity of at least 1 mL per 100 grams. In some embodiments, silicon oxide-containing porous solids comprise a water capacity of at least 5 mL per 100 grams. In some embodiments, silicon oxide-containing porous solids comprise a water capacity of at least 10 mL per 100 grams. In some embodiments, silicon oxide-containing porous solids comprise a water capacity of at least 20 mL per 100 grams. In some embodiments, silicon oxide-containing porous solids comprise a water capacity of at least 30 mL per 100 grams. In some embodiments, silicon oxide-containing porous solids comprise a water capacity of at least 35 mL per 100 grams. In some embodiments, silicon oxide-containing porous solids comprise a water capacity of at least 40 mL per 100 grams. In some embodiments, silicon oxide-containing porous solids comprise a water capacity of at least 45 mL per 100 grams. In some embodiments, silicon oxide-containing porous solids comprise a water capacity of at least 50 mL per 100 grams. In some embodiments, silicon oxide-containing porous solids comprise a water capacity of at least 75 mL per 100 grams. In some embodiments, silicon oxide-containing porous solids comprise a water capacity of at least 100 mL per 100 grams. In some embodiments, silicon oxide-containing porous solids comprise a water capacity of at least 125 mL per 100 grams. In some embodiments, silicon oxide-containing porous solids comprise a water capacity of at least 140 mL per 100 grams. In some embodiments, the silicon oxide-containing porous solids can provide a water capacity of from about 1 mL/100 g to about 140 mL per 100 grams, from about 1 mL per 100 grams to about 100 mL per 100 grams, from about 5 mL per 100 grams to about 50 mL per 100 grams, from about 10 mL per 100 grams to about 45 mL per 100 grams, from about 10 mL per 100 grams to about 40 mL per 100 grams, from about 10 mL per 100 grams to about 35 mL per 100 grams, from about 20 mL per 100 grams to about 35 mL per 100 grams, about 10 mL per 100 grams, about 15 mL/100 g, about 20 mL per 100 grams, about 25 mL per 100 grams, about 30 mL per 100 grams, or about 35 mL per 100 grams. In some embodiments, the silicon oxide-containing porous solids can provide a water capacity of from 1 mL/100 g to 140 mL per 100 grams, from 1 mL per 100 grams to 100 mL per 100 grams, from 5 mL per 100 grams to 50 mL per 100 grams, from 10 mL per 100 grams to 45 mL per 100 grams, from 10 mL per 100 grams to 40 mL per 100 grams, from 10 mL per 100 grams to 35 mL per 100 grams, from 20 mL per 100 grams to 35 mL per 100 grams, 10 mL per 100 grams, 15 mL/100 g, 20 mL per 100 grams, 25 mL per 100 grams, 30 mL per 100 grams, or 35 mL per 100 grams. The absorption capacity can also be expressed as weight percent of the silicon oxide-containing porous solids.

In some embodiments, the silicon oxide-containing porous solids can have a nicotine absorption capacity of from about 1 weight percent to about 100 weight percent of the silicon oxide-containing porous solids. In some embodiments, the silicon oxide-containing porous solids can have a nicotine absorption capacity of from 1 weight percent to 100 weight percent of the silicon oxide-containing porous solids. In some embodiments, a silicon-oxide containing porous solid comprises a nicotine absorption capacity of at least 1 weight percent of the silicon oxide-containing porous solid. In some embodiments, a silicon-oxide containing porous solid comprises a nicotine absorption capacity of at least 5 weight percent of the silicon oxide-containing porous solid. In some embodiments, a silicon-oxide containing porous solid comprises a nicotine absorption capacity of at least 10 weight percent of the silicon oxide-containing porous solid. In some embodiments, a silicon-oxide containing porous solid comprises a nicotine absorption capacity of at least 20 weight percent of the silicon oxide-containing porous solid. In some embodiments, a silicon-oxide containing porous solid comprises a nicotine absorption capacity of at least 30 weight percent of the silicon oxide-containing porous solid. In some embodiments, a silicon-oxide containing porous solid comprises a nicotine absorption capacity of at least 40 weight percent of the silicon oxide-containing porous solid. In some embodiments, a silicon-oxide containing porous solid comprises a nicotine absorption capacity of at least 50 weight percent of the silicon oxide-containing porous solid. In some embodiments, a silicon-oxide containing porous solid comprises a nicotine absorption capacity of at least 60 weight percent of the silicon oxide-containing porous solid. In some embodiments, a silicon-oxide containing porous solid comprises a nicotine absorption capacity of at least 70 weight percent of the silicon oxide-containing porous solid. In some embodiments, a silicon-oxide containing porous solid comprises a nicotine absorption capacity of at least 80 weight percent of the silicon oxide-containing porous solid. In some embodiments, a silicon-oxide containing porous solid comprises a nicotine absorption capacity of at least 90 weight percent of the silicon oxide-containing porous solid. In some embodiments, a silicon-oxide containing porous solid comprises a nicotine absorption capacity of at least 100 weight percent of the silicon oxide-containing porous solid.

In some embodiments, the silicon oxide-containing porous solids can have a water absorption capacity of from about 1 weight percent to about 140 weight percent of the silicon oxide-containing porous solids. In some embodiments, the silicon oxide-containing porous solids can have a water absorption capacity of from 1 weight percent to 140 weight percent of the silicon oxide-containing porous solids. In some embodiments, a silicon-oxide containing porous solid comprises a water absorption capacity of at least 1 weight percent of the silicon oxide-containing porous solid. In some embodiments, a silicon-oxide containing porous solid comprises a water absorption capacity of at least 5 weight percent of the silicon oxide-containing porous solid. In some embodiments, a silicon-oxide containing porous solid comprises a water absorption capacity of at least 10 weight percent of the silicon oxide-containing porous solid. In some embodiments, a silicon-oxide containing porous solid comprises a water absorption capacity of at least 20 weight percent of the silicon oxide-containing porous solid. In some embodiments, a silicon-oxide containing porous solid comprises a water absorption capacity of at least 30 weight percent of the silicon oxide-containing porous solid. In some embodiments, a silicon-oxide containing porous solid comprises a water absorption capacity of at least 40 weight percent of the silicon oxide-containing porous solid. In some embodiments, a silicon-oxide containing porous solid comprises a water absorption capacity of at least 50 weight percent of the silicon oxide-containing porous solid. In some embodiments, a silicon-oxide containing porous solid comprises a water absorption capacity of at least 60 weight percent of the silicon oxide-containing porous solid. In some embodiments, a silicon-oxide containing porous solid comprises a water absorption capacity of at least 70 weight percent of the silicon oxide-containing porous solid. In some embodiments, a silicon-oxide containing porous solid comprises a water absorption capacity of at least 80 weight percent of the silicon oxide-containing porous solid. In some embodiments, a silicon-oxide containing porous solid comprises a water absorption capacity of at least 90 weight percent of the silicon oxide-containing porous solid. In some embodiments, a silicon-oxide containing porous solid comprises a water absorption capacity of at least 100 weight percent of the silicon oxide-containing porous solid.

Silicon oxide-containing porous solids are generally hydrophilic, thus water soluble additives (e.g., nicotine) can preferentially be absorbed into pores of the silicon oxide-containing porous solids. In some embodiments, the hydrophilicity of the silicon oxide-containing porous solids can be modified. In some cases, silicon oxide-containing porous solids can be surface-modified to functionalize the surfaces of the pores and control absorption and release rates of various additives, including hydrophilic and hydrophobic additives. The hydrophilicity of the silicon oxide-containing porous solids can be selected to provide a desired sensorial experience when included in an oral product. For example, silicon oxide-containing porous solids can be modified to have greater or lesser hydrophilicity than an unmodified silicon oxide-containing porous solid.

In some embodiments, the particle size of the silicon oxide-containing porous solids can affect sensory aspects of the oral product, such as mouth feel. In an aspect, particle size is measured as the diameter of the particle. The particle size can, in some embodiments, also affect manufacturing and processing of the nicotine-containing silicon oxide-containing porous solids, as well as uniformity of nicotine distribution in oral product comprising the nicotine-containing silicon oxide-containing porous solids. In some cases, the silicon oxide-containing porous solids can have an average particle size of less than 200 micrometers. In some cases, the silicon oxide-containing porous solids comprise an average particle size of less than 175 micrometers. In some cases, the silicon oxide-containing porous solids comprise an average particle size of less than 150 micrometers. In some cases, the silicon oxide-containing porous solids comprise an average particle size of less than 100 micrometers. In some embodiments, the silicon oxide-containing porous solids can have an average particle size of at least 1 micrometer. In some embodiments, the silicon oxide-containing porous solids can have an average particle size of at least 5 micrometers. In some embodiments, the silicon oxide-containing porous solids can have an average particle size of at least 10 micrometers. In some embodiments, the silicon oxide-containing porous solids can have an average particle size of at least 25 micrometers. In some embodiments, the silicon oxide-containing porous solids can have an average particle size of at least 50 micrometers. In some embodiments, the silicon oxide-containing porous solids can have an average particle size of at least 75 micrometers. In some embodiments, the silicon oxide-containing porous solids can have an average particle size of between 10 micrometers and 200 micrometers. In some embodiments, the silicon oxide-containing porous solids can have an average particle size of between 10 micrometers and 150 micrometers. In some embodiments, the silicon oxide-containing porous solids can have an average particle size of between 10 micrometers and 100 micrometers. In some embodiments, the silicon oxide-containing porous solids can have an average particle size of between 10 micrometers and 75 micrometers. In some embodiments, the silicon oxide-containing porous solids can have an average particle size of between 100 micrometers and 200 micrometers. In some embodiments, the silicon oxide-containing porous solids can have an average particle size of between 50 micrometers and 200 micrometers. In other embodiments, the particles have a size of 100 micrometers or less, 80 micrometers or less, or 75 micrometers or less.

Nicotine can be incorporated into the pores of the silicon oxide-containing porous solid by mixing nicotine with the silicon oxide-containing porous solids. In some embodiments, mixing can be followed by a step of equilibrating the mixture. The silicon oxide-containing porous solid and nicotine can, in some embodiments, be prepared by a batch mixing process. In batch mixing, a pre-weighed amount of the porous solid can be loaded into the mixer. Next, a pre-weighed amount of nicotine or nicotine solution can be slowly added during mixing until a homogenous mixture is achieved. The nicotine and the silicon oxide-containing porous solid can be mixed in a suitable mixing device for any suitable length of time. Batch mixers useful in the methods provided herein can include, for example, ribbon blender, paddle blender, vertical screw blender, sigma mixer, planetary mixer, double cone blender, V-blenders, octagonal blender, plow mixer, double paddle mixer, rotary batch mixer, or other mixing apparatus depending on the desired batch size. In some cases, the silicon oxide-containing porous solid and nicotine can be mixed with a mixing implement rotating at a speed of less than 1000 rpm, less than 500 rpm, less than 250 rpm, less than 150 rpm, less than 100 rpm, less than 60 rpm, less than 30 rpm, or less than 10 rpm. In some cases, the silicon oxide-containing porous solid and nicotine can be mixed using a rotating and/or vibrating drum. In some cases, the silicon oxide-containing porous solid and nicotine can be mixed for at least 30 seconds, at least 1 minute, at least 3 minutes, at least 5 minutes, at least 10 minutes, at least 30 minutes, at least 60 minutes, or at least 120 minutes prior to incorporating a resulting porous solid-nicotine mixture into an oral product.

In some embodiments, the silicon oxide-containing porous solid and nicotine can be prepared by a continuous mixing process. In continuous mixing, the nicotine and silicon oxide-containing porous solid are continually charged into the mixer according to the desired formulation. Continuous mixers useful in the methods provided herein can include, for example, single screw extruder, twin screw extruder, twin screw continuous compounder, double auger mixer, paddle mixer, zig zag mixer, horizontal double shaft mixer, and rotating drum mixer. Mixing processes can generally be conducted at ambient conditions. In some embodiments, mixing can include nitrogen purging in order to minimize oxidation of the nicotine.

After mixing silicon oxide-containing porous solid and nicotine, the porous solid-nicotine mixture can be equilibrated in a sealed container. In some cases, the sealed container can be a bag (e.g., a poly bag). In some cases, the porous solid-nicotine mixture can be equilibrated for at least 1 minute, at least 10 minutes, at least 30 minutes, at least 1 hour, at least 2 hours, at least 4 hours, at least 6 hours, at least 8 hours, at least 10 hours, at least 12 hours, or at least 24 hours prior to use or incorporation into an oral product. In some cases, a porous solid-nicotine mixture can be further mixed or agitated during the equilibrating process. For example, a porous solid-nicotine mixture equilibrating in a poly bag can be agitated during the equilibrating process at a select time (e.g., 2 hours into the equilibrating process).

A silicon oxide-containing porous solid-nicotine mixture provided herein can be combined with other ingredients and/or packaging to make an oral product. In some cases, an oral product provided herein can include a molded body including at least one binder and a silicon oxide-containing porous solid-nicotine mixture.

In some embodiments, a method for incorporating nicotine into an oral product, is provided, comprising: (a) mixing nicotine with a silicon oxide-containing porous solid to produce a porous solid-nicotine mixture; (b) mixing the porous solid-nicotine mixture with one or more binders to form an oral product pre-molding mixture; and (c) molding the oral product pre-molding mixture into an oral product.

Silicon oxide-containing porous solid-nicotine mixtures provided herein can be used to stabilize nicotine for incorporation into an oral product. In some cases, an oral product provided herein can be produced by compression molding an oral product pre-molding mixture formed by mixing at least one or more binders and a silicon oxide-containing porous solid-nicotine mixture provided herein. The oral product pre-molding mixture can be produced by compression molding a dry mixture. A dry mixture, as the term is used herein, means that the components are introduced to the molding apparatus in a solid form, as opposed to a liquid or melted form. Dry ingredients, for example, can include cellulosic fiber having absorbed nicotine, sugar alcohols, gums, maltodextrin, polysaccharides, sweeteners, flavors, and/or antioxidants. In some cases, the oral product pre-molding mixture can be sintered to form an oral product. In some cases, the oral product pre-molding mixture can be injection molded to form an oral product. In some cases, the oral product pre-molding mixture can be extruded and cut to form one or more oral products.

An oral product provided herein can further include one or more flavorants, sweeteners, humectants, and/or plasticizers, such as the flavorants, sweeteners, humectants, and plasticizers discussed above. As noted above, flavorants, sweeteners, humectants, and/or plasticizers can be added to the nicotine as a diluent. In some cases, flavorants, sweeteners, humectants, and/or plasticizers can be added to a silicon oxide-containing porous solid-nicotine mixture provided herein after nicotine is absorbed. In some cases, flavorants, sweeteners, humectants, and/or plasticizers can be mixed with binder and a porous solid-nicotine mixture provided herein to form an oral product pre-molding mixture. Oral products provided herein can also include anti-oxidants and/or colorants.

The body of the oral product can have a variety of different shapes, some of which include disk, shield, rectangle, and square. According to certain embodiments, the body can have a length or width of between 5 mm and 100 mm and a thickness of between 1 mm and 30 mm. In some embodiments, the body comprises a length of at least 5 mm. In some embodiments, the body comprises a width of at least 5 mm. In some embodiments, the body comprises a thickness of at least 1 mm. In some embodiments, the body comprises a length of at least 10 mm. In some embodiments, the body comprises a width of at least 10 mm. In some embodiments, the body comprises a thickness of at least 5 mm. In some embodiments, the body comprises a length of at least 15 mm. In some embodiments, the body comprises a width of at least 15 mm. In some embodiments, the body comprises a thickness of at least 10 mm. In some embodiments, the body comprises a length of at least 25 mm. In some embodiments, the body comprises a width of at least 25 mm. In some embodiments, the body comprises a thickness of at least 20 mm. In some embodiments, the body comprises a length of at least 50 mm. In some embodiments, the body comprises a width of at least 50 mm. In some embodiments, the body comprises a thickness of at least 30 mm. In some embodiments, the body comprises a length of at least 75 mm. In some embodiments, the body comprises a width of at least 75 mm. In some embodiments, the body comprises a length of less than 100 mm. In some embodiments, the body comprises a width of less than 100 mm. In some embodiments, the body comprises a thickness of less than 30 mm. In some embodiments, the oral product can include edible films, gels, tabs, extruded products (e.g., extruded films, rod, etc.), shaped parts, consumable units, insoluble matrices, hollow shapes, chewable products, disintegratable products, etc. In some embodiments, the oral product comprises a shape-stable polymer.

The binder can be any suitable material that can hold a quantity of a silicon oxide-containing porous solid-nicotine mixture provided herein together as a single piece.

In some cases, the binder can be a water-soluble polymer such that a resulting oral product is dissolvable when exposed to saliva. For example, the binder can be a carbohydrate. In some cases, the binder includes a hydroxyl containing compound, a dextrin or dextrin derivative, carboxymethyl cellulose, hydroxypropyl cellulose, hydroxyethyl cellulose, hydroxypropyl methyl cellulose, methyl cellulose, starch, konjac, collagen, inulin, soy protein, whey protein, casein, wheat gluten, carrageenan, alginates, propylene glycol alginate, xanthan, dextrin, pullulan, curdlan, gellan, locust bean gum, guar gum, tara gum, gum tragacanth, pectin, agar, zein, karaya, gelatin, psyllium seed, chitin, chitosan, gum acacia, polyvinyl pyrrolidone, polyethylene oxide, polyvinyl alcohol, or a combination thereof. In some cases, the binder is selected from the group of guar gum, xanthan, cellulose, and combinations thereof. In some cases, the binder can include maltodextrin or other modified starches.

In some cases, the binder can be a mouth-stable polymer. Suitable mouth-stable polymer matrix can include polyurethane, silicon polymer, polyester, polyacrylate, polyethylene, poly(styrene-ethylene-butylene-styrene) (“SEBS”), poly(styrene-butadiene-styrene) (“SBS”), poly(styrene-isoprene-styrene)(“SIS”), and other similar thermoplastic elastomers, or any copolymer, mixture, or combination thereof.

In some cases, the binder can be a chewing gum base. A chewing gum base can include ingredients from the following categories: elastomers (such as couma macrocarpa, loquat, tunu, jelutong, chicle, styrene-butadiene rubber, butyl rubber, and polyisobutylene); resins (such as glycerol esters of gum, terpene resins, and/or polyvinyl acetate); waxes (such as paraffin or microcrystalline wax); fats (such as hydrogenated vegetable oils); emulsifiers (such as lecithin or glycerol monosterate); fillers (such as calcium carbonate or talc); antioxidants (e.g., BHT, BHA, tocopherol, ascorbyl palmitate). In some cases, a chewing gum base can include natural latexes, vegetable gums (e.g., chicle), spruce gum, and/or mastic gum.

In some embodiments, the silicon oxide-containing porous solid-nicotine mixture can be incorporated into a chewing gum oral product by known gum making methods. In an exemplary embodiment, the silicon oxide-containing porous solid-nicotine mixture can be added with, e.g., gum base, one or more sugar alcohols, one or more high intensity sweeteners, flavoring substances, or combinations thereof, to a Z-blade mixer and uniformly mixed.

In some embodiments, the silicon oxide-containing porous solid-nicotine mixture can be incorporated into compressed tablets, chewable tablets, and chewable/dissolvable products, by generally known tableting methods.

The oral products may be coated to, e.g., enhance flavor immediacy, improve product appearance, and/or affect shelf life. The concentration and amount of nicotine in the porous solid can be adjusted to provide the desired release rates from the final oral product.

Any references cited herein, including, e.g., all patents, published patent applications, and non-patent publications, are incorporated by reference herein in their entireties.

A number of embodiments of the invention have been described. Nevertheless, it will be understood that various modifications may be made without departing from the spirit and scope of the invention. Accordingly, other embodiments are within the scope of the following claims.

1. A composition comprising nicotine and a silicon oxide-containing porous solid.

2. The composition of claim 1, wherein the porous solid is amorphous.

3. The composition of claim 2, wherein the nicotine is absorbed within pores of the porous solid.

4. The composition of claim 1, wherein the porous solid comprises a material selected from a silica gel, a silica-containing polymer, an aluminosilicate, and combinations thereof.

5.-9. (canceled)

10. The composition of claim 1, wherein the porous solid has an average pore size of at least 0.5 nm.

11. The composition of claim 1, wherein the porous solid has a Brunauer-Emmett-Teller (BET) surface area of from about 1 m²/g to 1200 m²/g.

12. The composition of claim 1, wherein the nicotine is selected from free base nicotine and a nicotine salt.

13. The composition of claim 1, further comprising a diluent.

14. (canceled)

15. The composition of claim 1, wherein the porous solid has a nicotine absorption capacity or a water absorption capacity of 2 mL per 100 grams to 50 mL per 100 grams.

16. A method for stabilizing nicotine, comprising mixing nicotine with a silicon oxide-containing porous solid such that the nicotine absorbs into pores of the porous solid to form a porous solid-nicotine mixture.

17. The method of claim 16, wherein the porous solid comprises a material selected from a silica gel, a silica-containing polymer, an aluminosilicate, and combinations thereof.

18.-20. (canceled)

21. The method of claim 16, further comprising diluting nicotine with a diluent prior to mixing the nicotine with the porous solid.

22. The method of claim 16, wherein the diluent is selected from the group consisting of solvents, plasticizers, humectants, flavorants, and combinations thereof.

23. The method of claim 16, wherein the nicotine comprises at least 1 weight percent nicotine.

24. (canceled)

25. The method of claim 16, further comprising allowing the porous solid-nicotine mixture to equilibrate within a sealed container for at least 1 hour.

26.-29. (canceled)

30. A method for incorporating nicotine into an oral product, comprising:
a. mixing nicotine with a silicon oxide-containing porous solid to produce a porous solid-nicotine mixture;
b. mixing the porous solid-nicotine mixture with one or more binders to form an oral product pre-molding mixture; and
c. molding the oral product pre-molding mixture into an oral product.

31. The method of claim 30, further comprising diluting the nicotine with one or more diluents prior to mixing the nicotine with the porous solid to form the porous solid-nicotine mixture.

32. (canceled)

33. The method of claim 31, wherein the one or more diluents comprise:
(a) propylene glycol and the nicotine is diluted to a concentration of between 5% and 25% nicotine prior to mixing the nicotine with the porous solid to form the porous solid-nicotine mixture;
(b) water and the nicotine is diluted to a concentration of between 5% and 95% nicotine prior to mixing the nicotine with the porous solid to form the porous solid-nicotine mixture; or
(c) ethyl alcohol and the nicotine is diluted to a concentration of between 5% and 25% nicotine prior to mixing the nicotine with the porous solid to form the porous solid-nicotine mixture.

34.-35. (canceled)

36. The method of claim 30, wherein the ratio of nicotine to porous solid by weight is between 1:1000 and 1:1.

37.-38. (canceled)

39. The method of claim 30, wherein the binder is selected from the group consisting of dextrin or dextrin derivative, carboxymethyl cellulose, hydroxypropyl cellulose, hydroxyethyl cellulose, hydroxypropyl methyl cellulose, methyl cellulose, starch, konjac, collagen, inulin, soy protein, whey protein, casein, wheat gluten, carrageenan, alginates, propylene glycol alginate, xanthan, dextrin, pullulan, curdlan, gellan, locust bean gum, guar gum, tara gum, gum tragacanth, pectin, agar, zein, karaya, gelatin, psyllium seed, chitin, chitosan, gum acacia, polyvinyl pyrrolidone, polyethylene oxide, polyvinyl alcohol, guar gum, xanthan, cellulose, maltodextrin or other modified starch, polyurethane, silicon polymer, polyester, polyacrylate, polyethylene, poly(styrene-ethylene-butylene-styrene) (“SEBS”), poly(styrene-butadiene-styrene) (“SBS”), poly(styrene-isoprene-styrene)(“SIS”), couma macrocarpa, loquat, tunu, jelutong, chicle, styrene-butadiene rubber, butyl rubber, and polyisobutylene, glycerol esters of gum, terpene resins, polyvinyl acetate, paraffin, microcrystalline wax, hydrogenated vegetable oils, lecithin, glycerol monosterate, natural latexes, chicle, spruce gum, mastic gum, or a combination thereof.

40.-52. (canceled)
